(19) United States
(12) Patent Application Publication
(10) Pub. No.: US 2018/0333040 A1
(43) Pub. Date: Nov. 22, 2018

(54) COVER MEMBER OF BENDING OPERATION LEVER AND ENDOSCOPE INCLUDING COVER MEMBER OF BENDING OPERATION LEVER

(71) Applicant: OLYMPUS CORPORATION, Tokyo (JP)

(72) Inventors: Yudai HARADA, Tokyo (JP); Kiwamu FUJITANI, Tokyo (JP); Keisuke HATANO, Tokyo (JP)

(73) Assignee: OLYMPUS CORPORATION, Tokyo (JP)

(21) Appl. No.: 15/974,766

(22) Filed: May 9, 2018

Related U.S. Application Data
(60) Provisional application No. 62/508,468, filed on May 19, 2017.

(51) Int. Cl. A61B 1/005; A61B 1/00; G02B 23/24

(52) U.S. Cl. CPC A61B 1/0052; G02B 23/2476; A61B 1/00142

(57) ABSTRACT

A cover member of a bending operation lever includes a center portion fixed to a shaft section of a bending operation lever of an endoscope, an intermediate portion extended in a peripheral direction from the center portion and having first hardness, and a periphery portion extended in the peripheral direction of the intermediate portion, having second hardness higher than the first hardness, and fixed to the endoscope.

### BACKGROUND OF THE INVENTION

### Field of the Invention

The present invention relates to a cover member of a bending operation lever that performs tilting operation to realize a bending motion of a bending section and an endoscope including the cover member of the bending operation lever.

### Description of Related Art

As a device for observing a region hardly observed by normal means, for example, an inside of a living organism or a structure, an endoscope insertable into inside of a subject has been widely put to practical use in, for example, a medical field or an industrial field.

In an insertion section of the conventional endoscope of this type, in general, a bending section is provided in order to improve insertability into the subject and observability. The bending section is bent by a bending operation device provided in an operation section.

As the bending operation device in the conventional endoscope, for example, as disclosed by Japanese Patent Application Laid-Open Publication No. 2004-321612, a bending operation device including a joystick-type bending operation lever that performs tilting operation to realize a bending motion of the bending section is well-known.

In general, reprocess treatment (cleaning and disinfection and sterilization treatment) is performed on an endoscope, in particular, an endoscope used in the medical field every time the endoscope is used. Therefore, the endoscope normally includes a watertight structure.

On the other hand, in the endoscope including the configuration explained above, that is, the joystick-type bending operation lever, the bending operation lever is projected toward an outside of the operation section. The bending operation lever is operated to be tilted. Therefore, near the bending operation lever, it is necessary to take measures to secure water-tightness of the operation section of the endoscope while allowing a lever tilting motion.

Therefore, some conventional endoscope including the joystick-type bending operation lever is configured to, for example, water-tightly cover an outer surface of the bending operation lever with a cover member formed of an elastic member such as rubber.

In the case of this configuration, in particular, in a part near an attachment section to the operation section of the bending operation lever, water-tightness of the operation section is secured by, for example, holding an end portion of the cover member with a plurality of components made of a metal material or the like.

### SUMMARY OF THE INVENTION

A cover member of a bending operation lever according to an aspect of the present invention includes: a center portion fixed to a bending operation lever of an endoscope; an intermediate portion extended in a peripheral direction from the center portion and having first hardness; and a periphery portion extended in the peripheral direction of the intermediate portion, having second hardness higher than the first hardness, and fixed to the endoscope.

An endoscope according to another aspect of the present invention includes: a bending section provided in an insertion section; a bending operation lever provided in an operation section; and the cover member of the bending operation lever.

Benefits of the present invention will become clearer from the following detailed explanation.

### BRIEF DESCRIPTION OF THE DRAWINGS

FIG. 1 is a schematic configuration diagram schematically showing an exterior of an endoscope including a cover member of a bending operation lever according to an embodiment of the present invention;

FIG. 2 is a main part exterior view of a part of an operation section in the endoscope shown in FIG. 1 viewed from a side; and

FIG. 3 is a schematic configuration diagram mainly showing a configuration of the cover member of the bending operation lever in the endoscope shown in FIG. 1.

### DETAILED DESCRIPTION OF THE PREFERRED EMBODIMENT

An embodiment of the present invention is explained below with reference to the drawings. The respective drawings used for the following explanation are schematically shown. In order to show respective components in recognizable sizes on the drawings, dimensional relations, scales, and the like of respective members are sometimes differentiated and shown for each of the respective components. Therefore, the present invention is not limited to only forms shown in the figures concerning numbers of the respective components, shapes of the respective components, ratios of sizes of the respective components, relative positional relations of the respective components, and the like described in the respective drawings. In the following explanation, upward and downward directions on a paper surface of a figure are sometimes explained as upper parts and lower parts of the components.

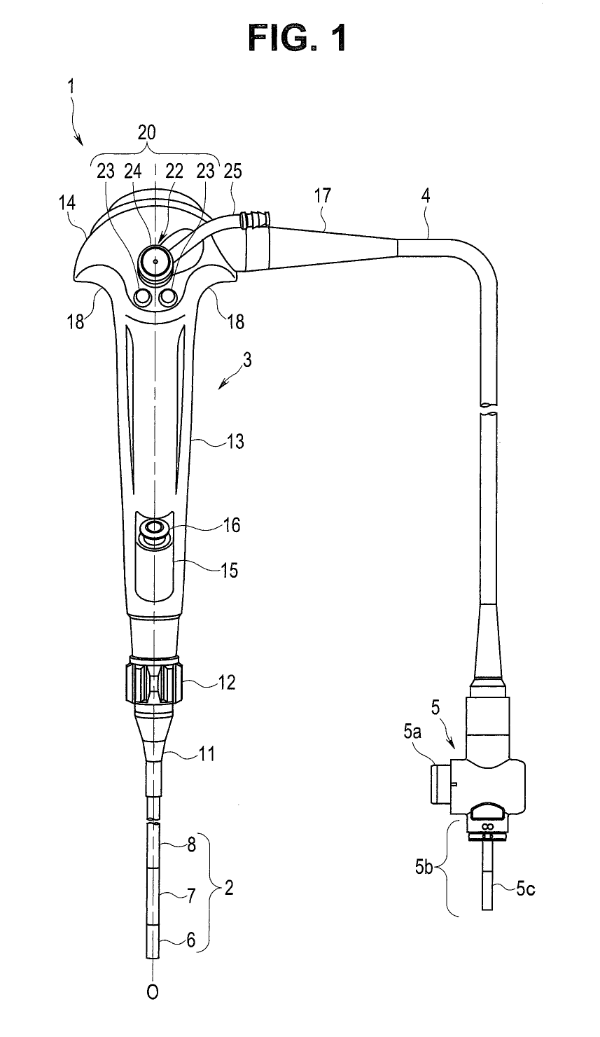

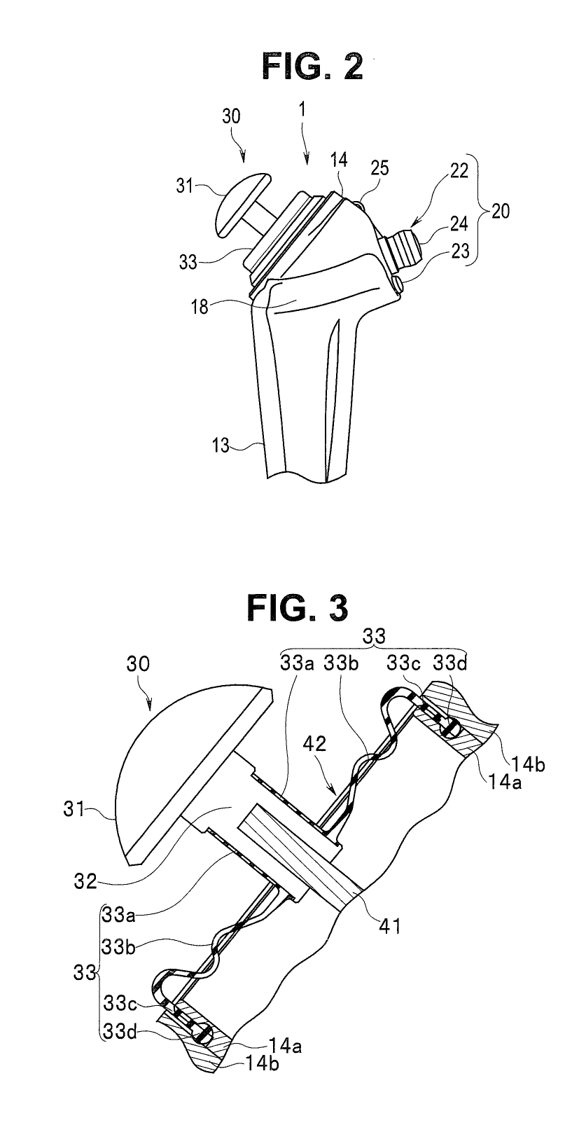

First, an endoscope including a cover member of a bending operation lever according to an embodiment of the present invention is explained below with reference to the drawings. FIG. 1 is a front view showing an exterior configuration of the endoscope. FIG. 2 is a left side view showing an exterior configuration of an operation section of the endoscope. FIG. 3 is a partial sectional view mainly showing a configuration of the cover member of the bending operation lever.

As shown in FIG. 1 and the like, an endoscope 1 is an electronic endoscope. The endoscope 1 mainly includes an insertion section 2 formed in an elongated tubular shape, an operation section 3 concatenated at a proximal end of the insertion section 2, a universal cord 4, which is an endoscope cable extended from the operation section 3, and an endoscope connector 5 disposed at a distal end of the universal cord 4.

In the insertion section 2 of the endoscope 1, a distal end portion 6, a bending section 7, and a flexible tube section 8 are concatenated in order from a distal end side. The insertion section 2 is configured in a tubular shape having flexibility.

In the distal end portion 6 of the insertion section 2, although not shown in FIG. 1 and the like, an objective optical system, an image pickup unit incorporating an image sensor such as a CCD or a CMOS, an illumination optical system configured to irradiate illumination light transmitted by a light guide bundle, a channel pipe configured to connect and hold a treatment instrument channel, and the like are disposed.

The bending section 7 of the insertion section 2 is configured to be capable of actively bending in all circumferential directions around an insertion axis O including upward, downward, left, and right directions (UP-DOWN/RIGHT-LEFT) according to an operation input by a surgeon, a user, or the like to the operation section 3.

The flexible tube section 8 of the insertion section 2 is configured by a tubular member having flexibility capable of passively bending. An image pickup cable, a light guide bundle, a treatment instrument insertion channel, and an air/water feeding tube (none of which is shown in FIG. 1 and the like) are inserted through an inside of the flexible tube section 8.

The operation section 3 of the endoscope 1 includes a bend preventing section 11 connected to the flexible tube section 8 in a state in which the bend preventing section 11 covers a proximal end of the flexible tube section 8, an insertion section rotary dial 12 capable of adjusting a rotating position around the insertion axis O of the insertion section 2 provided on a proximal end side of the bend preventing section 11, a grasping section 13 concatenated to a proximal end side of the insertion section rotary dial 12 and graspable by a hand of the user or the like, and an operation section main body 14 concatenated to a proximal end side of the grasping section 13.

Note that, in this embodiment, for example, a direction around the insertion axis O serving as a longitudinal axis in the operation section 3 is defined on the basis of a state in which the user or the like grasps the grasping section 13. More specifically, in the operation section 3, front, rear, left, and right directions (a front surface, a rear surface, left and right side surfaces, etc.) based on the user or the like who grasps the grasping section 13 are defined.

The grasping section 13 is formed in a bilaterally symmetrical shape with respect to the insertion axis O. The user or the like is capable of grasping the grasping section 13 with either a left hand or a right hand.

A treatment instrument insertion section 15 is provided on a front surface on a distal end side of the grasping section 13. The treatment instrument insertion section 15 includes a treatment instrument insertion opening 16 into which not-shown various treatment instruments are inserted.

On an inside of the operation section 3, a treatment instrument insertion channel is caused to communicate with the treatment instrument insertion opening 16 via a branching member (none of which is shown in FIG. 1 and the like). In the treatment instrument insertion section 15, a lid member for closing the treatment instrument insertion opening 16, for example, a disposable forceps plug (not shown in FIG. 1 and the like) is detachably provided.

The operation section main body 14 is mainly configured by a hollow member formed in a substantially partial spherical shape swelled to left and right sides and a front on the proximal end side of the grasping section 13. Operation buttons 20 for executing a suction function, various optical functions, and the like of the endoscope 1 are disposed on a front surface side of the operation section main body 14.

The operation buttons 20 include, for example, a disposable suction valve 22 detachably attached to the operation section main body 14 and two button switches 23 capable of selectively allocating any function, for example, a release button out of various functions concerning the endoscope 1.

Note that the suction valve 22 includes a suction button 24 functioning as an operation input member and a tube connecting section 25 to which a suction tube extended from a not-shown endoscope suction device, which is an external device, is connected.

On a rear surface side of the operation section main body 14, as shown in FIG. 2, a bending operation lever 30 functioning as a bending operation member for performing bending operation on the bending section 7 is disposed.

The universal cord 4 is extended from one side portion (e.g., a left side portion) of the operation section main body 14 via a cable bend preventing section 17.

As shown in FIG. 1, a left-right shape of the operation section main body 14 is a swelling shape bilaterally symmetrical with respect to the insertion axis O. On left and right side surfaces on a distal end side of the operation section main body 14, concave sections for guide 18 for guiding an index finger or the like of the user, who grasps the grasping section 13, to the operation buttons 20 are respectively formed.

The universal cord 4 leads to the operation section 3 from the distal end portion 6 side through the inside of the insertion section 2. The universal cord 4 is a composite cable through which various signal lines including the image pickup cable extended from the operation section 3, a light guide bundle, and a tube for air/water feeding into which fluid for air/water feeding is caused to flow (none of which is shown in FIG. 1 and the like) are inserted.

The endoscope connector 5 provided at the end portion of the universal cord 4 includes an electric connector section 5a provided in a side surface portion and a light source connector section 5b connected to a not-shown light source device, which is an external device.

Note that a connector of an electric cable extended from a not-shown video processor, which is an external device, is detachably connected to the electric connector section 5a. A light guide connector section 5c, in which the light guide bundle is housed, is disposed in the light source connector section 5b.

A configuration of the bending operation lever 30 provided in the operation section main body 14 is explained in detail below mainly with reference to FIG. 3.

The bending operation lever 30 is an operation member configured to be capable of tilting in all directions around the insertion axis O. The bending operation lever 30 is provided with a part projecting from the inside of the operation section 3 toward an outside of the operation section 3. That is, the bending operation lever 30 in this embodiment is a so-called joystick-type lever member.

As shown in FIG. 3, the bending operation lever 30 is mainly configured by a finger placing section 31, a shaft section 32, and a cover member 33.

The finger placing section 31 is a part provided at a projecting end portion of the bending operation lever 30. A thumb or the like of the user comes into contact with the finger placing section 31 when tilting operation by the user is performed. The user brings the thumb or the like into contact with the finger placing section 31 and presses the finger placing section 31 in a desired direction to thereby perform the tiling operation of the bending operation lever 30.

Therefore, the finger placing section 31 is disposed in a position projecting outward from a surface of the operation section 3 such that, when the user grasps the grasping section 13, the finger placing section 31 can be operated by the thumb of the hand of the user who grasps the grasping section 13.

The shaft section 32 is a bar-like member formed in a substantially columnar shape. The finger placing section 31 is fixed to one end of the shaft section 32. A shaft member 41 (see FIG. 3) extended from a bending operation mechanism (not shown in FIG. 1 and the like) provided on an inside of the operation section main body 14 of the operation section 3 are coupled on the same axis at the other end of the shaft section 32. Consequently, when the user tilts the bending operation lever 30 in a desired direction, the bending operation mechanism (not shown in FIGS. 1 and the like) acts. The bending section 7 bends in a direction corresponding to an operating direction of the bending operation lever 30.

In the operation section main body 14 of the operation section 3, a hole section 42 is formed in a part where the shaft section 32 is provided. The hole section 42 is provided to secure a moving region where the shaft section 32 of the bending operation lever 30 tilts. Therefore, the shaft section 32 is disposed substantially in the center of the hole section 42. The shaft section 32 tilts around a center axis of the hole section 42.

The cover member 33 is a waterproof member for water-tightly sealing the hole section 42 from the outside while allowing a tilting motion of the shaft section 32 around the shaft section 32 projected from the operation section 3. The cover member 33 is configured by an elastic member having flexibility such as rubber as a whole.

The cover member 33 includes a center portion 33a, an intermediate portion 33b, and a periphery portion 33c.

In detail, the center portion 33a of the cover member 33 is a part fixed to the shaft section 32 of the bending operation lever 30 provided in the operation section main body 14 of the operation section 3 of the endoscope 1.

That is, the center portion 33a water-tightly covers a substantially entire outer surface (an outer surface from a part near the finger placing section 31 to a part near the other end) of the shaft section 32. In the center portion 33a, the cover member 33 is water-tightly fixed to the outer surface of the shaft section 32 by fixing means such as adhesion fixing.

The intermediate portion 33b of the cover member 33 is a part extended in a peripheral direction from the center portion 33a. The intermediate portion 33b has first hardness (explained in detail below).

That is, the intermediate portion 33b is a part extended in the peripheral direction from the center portion 33a (from a part near the other end of the shaft section 32). The intermediate portion 33b covers a region of the hole section 42, that is, the entire moving region of the shaft section 32 where the shaft section 32 tilts. In order to allow the tilting motion of the shaft section 32, the intermediate portion 33b forms a mountain fold portion and a valley fold portion, a cross section of the intermediate portion 33b is formed in a zigzag shape, and the intermediate portion 33b is formed in a substantially annular sheet shape as a whole.

The intermediate portion 33b has flexibility so as for the tilting motion of the shaft section 32 to be allowed and follows the tilting motion. Hardness of the intermediate portion 33b is referred to as first hardness.

The periphery portion 33c of the cover member 33 is a part extended in the peripheral direction of the intermediate portion 33b and fixed to the operation section main body 14 of the operation section 3 of the endoscope 1. The periphery portion 33c has second hardness higher than the first hardness (explained in detail below).

That is, the periphery portion 33c is water-tightly fixed while being held between predetermined two components 14a and 14b among a plurality of components that are a part of the operation section main body 14 of the operation section 3 and configure the operation section main body 14. Therefore, a seal ring portion 33d formed in an O-shaped ring shape is integrally formed at an end part of the periphery portion 33c.

The seal ring portion 33d is in pressurized contact with and fixed between the predetermined two components 14a and 14b in the operation section main body 14. Consequently, water-tightness is secured between the two components 14a and 14b.

Note that the periphery portion 33c and the seal ring portion 33d are parts held between the predetermined two components 14a and 14b as explained above. However, in FIG. 3, the periphery portion 33c and the seal ring portion 33d are shown in original shapes. However, in a state in which the periphery portion 33c and the seal ring portion 33d are incorporated in the endoscope 1, the periphery portion 33c and the seal ring portion 33d are actually held between the predetermined two components 14a and 14b and have a crashed shape.

Hardness of the periphery portion 33c is referred to as second hardness. The second hardness is higher than the first hardness in the intermediate portion 33b.

Therefore, since the cover member 33 is configured as explained above, the intermediate portion 33b has flexibility with the first hardness and allows the tilting motion of the shaft section 32.

On the other hand, the periphery portion 33c is prevented from being easily deformed with the second hardness higher than the first hardness.

Therefore, for example, even if the shaft section 32 is tilted and the cover member 33 is pulled in a tilting direction of the shaft section 32, the intermediate portion 33b is flexibly deformed and, on the other hand, the periphery portion 33c is not easily deformed. Therefore, the periphery portion 33c can always retain the water-tightness between the predetermined two components 14a and 14b.

Note that, in the cover member 33, in order to form the intermediate portion 33b with the first hardness and form the periphery portion 33c with the second hardness higher than the first hardness, more specifically, a configuration explained below only has to be adopted.

For example, in the cover member 33, the intermediate portion 33b is formed of an elastic member having low rigidity and a low modulus of elasticity and easily deformed, for example, a rubber member. The periphery portion 33c is formed of an elastic member (a rubber member, etc.) having high rigidity and a high modulus of elasticity and hardly deformed. In this case, the intermediate portion 33b and the periphery portion 33c are formed by adhesion means such as welding.

As another form, the cover member 33 may be formed of the same material as a whole. The intermediate portion 33b may be formed in, for example, a thin-film sheet shape to reduce the rigidity. The periphery portion 33c may be formed thicker than the intermediate portion 33b to increase the rigidity.

As still another form, in the cover member 33, the intermediate portion 33b is formed of a rubber member having low rigidity and a low modulus of elasticity and easily deformed. The periphery portion 33c is formed of a tabular member made of resin (a plastic material, etc.), metal, or the like having high rigidity. In this case as well, the intermediate portion 33b and the periphery portion 33c are integrally formed by adhesion means such as welding. Note that, in the case of this configuration, the seal ring portion 33d at the end part of the periphery portion 33c is desirably formed of a rubber member.

As a different form, in the cover member 33, the intermediate portion 33b may be formed of a rubber member having low rigidity and a low modulus of elasticity and easily deformed. The periphery portion 33c may be formed of the same rubber member. A tabular member made of resin (a plastic material, etc.), metal, or the like having high rigidity may be embedded and formed on an inside of the periphery portion 33c. Note that, in the case of this configuration, the seal ring portion 33d at the end part of the periphery portion 33c is desirably formed of only the rubber member.

As explained above, according to the embodiment, in the cover member 33 of the bending operation lever 30, the center portion 33a is surely water-tightly fixed to the shaft section 32, the intermediate portion 33b is formed of a soft material having low rigidity (the first hardness), and the periphery portion 33c is formed of a harder material having higher rigidity than the intermediate portion 33b (the second hardness higher than the first hardness).

By adopting such a configuration, the cover member 33 in this embodiment can surely allow a necessary tilting motion of the shaft section 32 in the intermediate portion 33b and, at the same time, surely secure water-tightness of the endoscope 1 in the periphery portion 33c.

The intermediate portion 33b is configured to have low rigidity and a low modulus of elasticity and to be easily deformed. Therefore, it is possible to tilt the shaft section 32 of the bending operation lever 30 with a smaller operation amount of force. Therefore, it is possible to contribute to improvement of operability of the endoscope 1.

Further, the periphery portion 33c is configured to have high rigidity and a high modulus of elasticity and to be hardly deformed. Therefore, it is possible to reduce a deformation amount during the tilting motion of the shaft section 32 of the bending operation lever 30. Therefore, in the part, an unnecessary gap or the like is prevented from occurring between the cover member 33 and the operation section main body 14. Therefore, it is possible to always secure sure water-tightness.

The present invention is not limited to the embodiment explained above. It goes without saying that various modifications and application can be carried out within a range not departing from the spirit of the invention. Further, the embodiment includes inventions in various stages. By appropriately combining a plurality of disclosed constituent elements, various inventions can be extracted. For example, when the problem to be solved by the invention can be solved and the effect of the invention can be obtained even if several constituent elements are deleted from all the constituent elements explained in the embodiment, a configuration from which the constituent elements are deleted can be extracted as an invention. Further, the constituent elements in different embodiments may be combined as appropriate. The present invention is not limited by an embodiment specific to the invention except that the present invention is limited by the appended claims.

What is claimed is:

1. A cover member of a bending operation lever comprising:
a center portion fixed to a shaft section of a bending operation lever of an endoscope;
an intermediate portion extended in a peripheral direction from the center portion and having first hardness; and
a periphery portion extended in the peripheral direction of the intermediate portion, having second hardness higher than the first hardness, and fixed to the endoscope.

2. The cover member of the bending operation lever according to claim 1, wherein the intermediate portion and the periphery portion are formed of a rubber material.

3. The cover member of the bending operation lever according to claim 1, wherein
the intermediate portion is formed of a rubber material, and
the periphery portion is formed of a resin material.

4. The cover member of the bending operation lever according to claim 1, wherein
the intermediate portion is formed of a rubber material, and
the periphery portion is formed of a metal material.

5. The cover member of the bending operation lever according to claim 1, wherein the intermediate portion is formed in a flexible sheet shape.

6. An endoscope comprising:
a bending section provided in an insertion section;
a bending operation lever provided in an operation section; and
the cover member of the bending operation lever according to claim 1.
